(19) United States
(12) Patent Application Publication
(10) Pub. No.: US 2015/0141809 A1
(43) Pub. Date: May 21, 2015

(54) DEVICES, SYSTEMS, AND METHODS FOR NAVIGATING A BIOPSY TOOL TO A TARGET LOCATION AND OBTAINING A TISSUE SAMPLE USING THE SAME

(71) Applicant: COVIDIEN LP, Mansfield, MA (US)

(72) Inventors: DAVID M. COSTELLO, DELANO, MN (US); THOMAS P. CROWLEY, LINO LAKES, MN (US); THOMAS D. MAGNUSON, PLYMOUTH, MN (US)

(21) Appl. No.: 14/488,754

(22) Filed: Sep. 17, 2014

Related U.S. Application Data
(60) Provisional application No. 61/906,762, filed on Nov. 20, 2013.

(51) Int. Cl. A61B 10/04; A61B 10/02; A61B 5/06

(52) U.S. Cl. CPC A61B 10/04; A61B 5/061; A61B 10/0275
USPC 600/424

(57) ABSTRACT

A biopsy tool includes an elongated flexible body defining a distal end and a distal biopsy member disposed at the distal end of the elongated flexible body. The biopsy member incorporates a sensor assembly configured to enable detection of a location of the sensor assembly within a patient's airways. The biopsy member has a tissue-receiving portion defining a window and including first and second longitudinally-extending faces disposed on either side of the window. The faces are angled inwardly and towards one another to define an acute interior angle therebetween. Each face defines a sharpened cutting edge. The sharpened cutting edges are disposed on either side of the window. The faces are positioned such that the sharpened cutting edges increasingly approximate one another in the proximal-to-distal direction and culminate at an apex point.

### CROSS-REFERENCE TO RELATED APPLICATIONS

This application claims the benefit of, and priority to, U.S. Provisional Patent Appln. No. 61/906,762, filed on Nov. 20, 2013, the entire contents of which are incorporated herein by reference.

### BACKGROUND

1. Technical Field

The present disclosure relates to biopsy sampling and, more particularly, to devices, systems, and methods for navigating a biopsy tool to a target location and obtaining a tissue sample using the biopsy tool.

2. Description of Related Art

A bronchoscope is inserted into a patient's airways through the patient's nose or mouth. A typical bronchoscope includes an elongated flexible tube having an illumination assembly for illuminating the region distal to the bronchoscope's tip, an imaging assembly for providing a video image from the bronchoscope's tip, and a working channel through which instruments, e.g., diagnostic instruments such as biopsy tools and/or therapeutic instruments such as ablation probes, can be inserted.

Bronchoscopes are limited in how far they may be advanced through the airways due to their size. Where the bronchoscope is too large to reach a target location deep in the lungs, a locatable guide (“LG”) enveloped by a sheath is often utilized to navigate from the end of the bronchoscope to the target location. That is, the LG, together with a navigation system, enables the position and orientation of the LG to be tracked as the LG is advanced through the airways.

In use, the LG/sheath combination is inserted through the working channel of the bronchoscope and into the patient's airways. Once the LG has been navigated to the target location, aided by the position and orientation tracking provided by the navigation system, the LG is retracted through the sheath, leaving the sheath in position. With the LG retracted, the sheath is often referred to as an extended working channel (“EWC”) because it effectively functions as an extension of the working channel of the bronchoscope.

Once the LG has been retracted from the EWC, the EWC may be used as an avenue for guiding working tools, e.g., biopsy tools, ablation probes, etc., to the target location. However, once the LG is removed from the EWC, tracking is no longer provided and, thus, the operator is operating blind, relying on the EWC to remain fixed at the target location. Repositioning of the working tool at the target location is likewise required to be performed without guidance.

### SUMMARY

As used herein, the term “distal” refers to the portion that is being described which is further from a user, while the term “proximal” refers to the portion that is being described which is closer to a user. Further, to the extent consistent, any of the aspects and features detailed herein may be used in conjunction with any or all of the other aspects and features detailed herein.

A biopsy tool provided in accordance with the present disclosure includes an elongated flexible body defining a distal end and a distal biopsy member disposed at the distal end of the elongated flexible body. The distal biopsy member incorporates a sensor assembly including at least one location sensor configured to enable detection of a location of the sensor assembly within a patient's airways. The distal biopsy member has a tissue-receiving portion defining a window and including first and second longitudinally-extending faces disposed on either side of the window. The faces are angled inwardly and towards one another to define an acute interior angle therebetween. Each face defines a sharpened cutting edge. The sharpened cutting edges are disposed on either side of the window. The faces are positioned such that the sharpened cutting edges increasingly approximate one another in the proximal-to-distal direction and culminate at an apex point.

In aspects, the tissue-receiving portion of the distal biopsy member is recessed relative to a body of the distal biopsy member to define proximal and distal shoulders at proximal and distal ends of the tissue-receiving portion.

In aspects, the distal biopsy member is configured to connect to a vacuum source for applying suction adjacent the window.

Another biopsy tool provided in accordance with the present disclosure includes, similarly as above, an elongated flexible body defining a distal end and a distal biopsy member disposed at the distal end of the elongated flexible body. The distal biopsy member incorporates a sensor assembly including at least one location sensor configured to enable detection of a location of the sensor assembly within a patient's airways. The distal biopsy member includes an outer member defining a hollow configuration and an inner member including a shaft and a distal end cap. The inner member is slidable relative to the outer member between a retracted position, wherein the shaft is disposed within the outer member and the distal end cap is at least partially disposed within outer member, and an extended position, wherein the distal end cap and the shaft extend distally from the outer member such that the distal end cap is distally-spaced from the outer member. The distal end cap defines a sharpened distal tip configured to facilitate tissue penetration and a sharpened proximal rim configured to facilitate cutting tissue disposed between the distal end cap and the outer member upon return of the inner member towards the retracted position.

In aspects, the inner member is rotatable relative to the outer member to further facilitate cutting tissue disposed between the distal end cap and the outer member upon return of the inner member towards the retracted position.

In aspects, the distal end cap defines a hollow interior configured to receive a portion of a tissue sample therein.

Yet another biopsy tool provided in accordance with the present disclosure includes, similarly as above, an elongated flexible body defining a distal end and a distal biopsy member disposed at the distal end of the elongated flexible body. The distal biopsy member incorporates a sensor assembly including at least one location sensor configured to enable detection of a location of the sensor assembly within a patient's airways. The distal biopsy member includes an outer member and an inner member. The outer member includes a head portion defining a distal end cap and having a mouth extending through a lateral wall of the head portion towards the distal end cap. The inner member is disposed within the outer member and defines an open distal end having a sharpened rim positioned adjacent the mouth of the outer member.

In aspects, the inner member is fixed relative to the outer member. Alternatively, the inner member may be rotatable relative to the outer member.

In aspects, the distal biopsy member is configured to connect to a vacuum source for applying suction adjacent the open distal end of the inner member.

Still yet another biopsy tool provided in accordance with the present disclosure includes, similarly as above, an elongated flexible body defining a distal end and a distal biopsy member disposed at the distal end of the elongated flexible body. The distal biopsy member incorporates a sensor assembly including at least one location sensor configured to enable detection of a location of the sensor assembly within a patient's airways. The distal biopsy member includes an outer member and an inner member. The outer member includes a head portion defining a distal end cap and having a first mouth extending through a lateral wall of the head portion towards the distal end cap. The inner member is disposed within the outer member. The inner member defines a second mouth extending through a lateral wall of the inner member and positioned adjacent the first mouth. The inner member further includes a sharpened rim disposed about the second mouth.

In aspects, the inner member is fixed relative to the outer member. Alternatively, the inner member may be rotatable relative to the outer member to move the first and second mouths at least between an aligned position, a partially overlapping position, and an occluded position.

In aspects, the distal biopsy member is configured to connect to a vacuum source for applying suction adjacent the second mouth of the inner member.

### BRIEF DESCRIPTION OF THE DRAWINGS

Various aspects and features of the present disclosure are described hereinbelow with references to the drawings, wherein:

FIG. 1 is a perspective view of a system provided in accordance with the present disclosure configured for navigating a biopsy tool to a target location and obtaining a tissue sample using the biopsy tool;

FIG. 2 is a perspective view of the distal end of one embodiment of a biopsy tool provided in accordance with the present disclosure and configured for use with the system of FIG. 1;

FIG. 3 is a perspective view of the distal end of another embodiment of a biopsy tool provided in accordance with the present disclosure and configured for use with the system of FIG. 1;

FIG. 4A is a perspective view of the distal end of another embodiment of a biopsy tool provided in accordance with the present disclosure and configured for use with the system of FIG. 1;

FIG. 4B is a perspective view of the distal end of yet another embodiment of a biopsy tool provided in accordance with the present disclosure and configured for use with the system of FIG. 1;

FIG. 5A is a perspective view of the distal end of still another embodiment of a biopsy tool provided in accordance with the present disclosure and configured for use with the system of FIG. 1;

FIG. 5B is a perspective view of the distal end of still yet another embodiment of a biopsy tool provided in accordance with the present disclosure and configured for use with the system of FIG. 1;

FIG. 6 is a perspective view of an embodiment of a sensor configured for use with any of the biopsy tools of the present disclosure;

FIG. 7 is a perspective view of another embodiment of a sensor configured for use with any of the biopsy tools of the present disclosure;

FIG. 8 is a perspective view of yet another embodiment of a sensor configured for use with any of the biopsy tools of the present disclosure; and

FIG. 9 is an exploded, perspective view of a transmitter mat configured for use with the system of FIG. 1 for tracking a biopsy tool through a patient's airways.

### DETAILED DESCRIPTION

Devices, systems, and methods for navigating a biopsy tool to a target location and obtaining a tissue sample using the biopsy tool are provided in accordance with the present disclosure and described in detailed below. The various biopsy tools of the present disclosure, for example, each generally include a flexible body, a biopsy member disposed at the distal end of the flexible body, and a sensor assembly integrated into the biopsy tool and positioned adjacent the biopsy member. The biopsy member is configured to facilitate obtaining a tissue sample. The sensor assembly enables determination of the current location of the biopsy member, thus facilitating navigation of the biopsy member to target tissue and/or manipulation of the biopsy member relative to target tissue. Detailed embodiments of such devices, systems incorporating such devices, and methods using the same as described below. However, these detailed embodiments are merely examples of the present disclosure, which may be embodied in various forms.

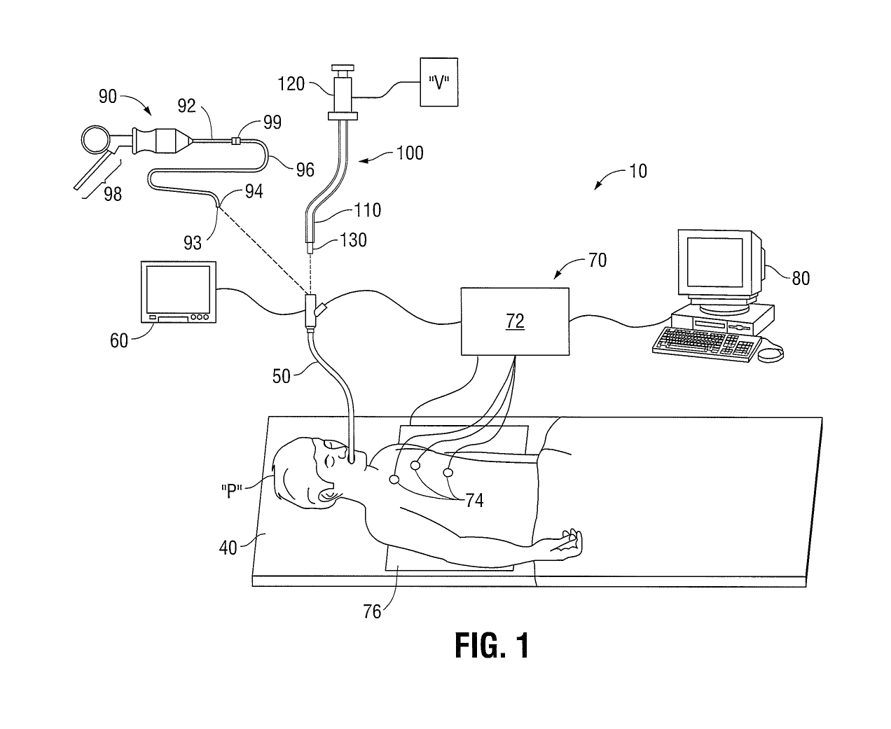

With reference to FIG. 1, a system provided in accordance with the present disclosure and configured for planning a pathway to target tissue (planning phase), navigating a positioning assembly to the target tissue (navigation phase), and navigating a biopsy tool to the target tissue to obtain a tissue sample from the target tissue using the biopsy tool (biopsy phase) is shown generally identified by reference numeral 10. System 10 generally includes an operating table 40 configured to support a patient “P;” a bronchoscope 50 configured for insertion through the patient's mouth into the patient's airways; monitoring equipment 60 coupled to bronchoscope 50 for displaying video images received from bronchoscope 50; a tracking system 70 including a tracking module 72, a plurality of reference sensors 74, and a transmitter mat 76; a computer 80 including software and/or hardware used to facilitate pathway planning, identification of target tissue, and navigation to target tissue; a positioning assembly 90 including an LG 92 and an EWC 96; and a biopsy tool 100 operable to obtain a tissue sample, e.g., for subsequent diagnostic testing. The planning and navigation phases will initially be detailed below, followed by a detailed description of biopsy tools provided in accordance with the present disclosure and use of such biopsy tools in conjunction with system 10 in performing the biopsy phase.

With respect to the planning phase, computer 80 utilizes computed tomographic (CT) image data for generating and viewing a three-dimensional model of the patient's airways, enables the identification of target tissue on the three-dimensional model (automatically, semi-automatically or manually), and allows for the selection of a pathway through the patient's airways to the target tissue. More specifically, the CT scans are processed and assembled into a three-dimensional CT volume, which is then utilized to generate a three-dimensional model of the patient's airways. The three-dimensional model may be displayed on a display monitor associated with computer 80, or in any other suitable fashion. Using computer 80, various views of the three-dimensional model may be provided and/or the three-dimensional model may be manipulated to facilitate identification of target tissue on the three-dimensional model and selection of a suitable pathway through the patient's airways to access the target tissue. Once selected, the pathway is saved for use during the navigation phase(s).

Continuing with reference to FIG. 1, patient “P” is shown lying on operating table 40 with bronchoscope 50 inserted through the patient's mouth and into the patient's airways. Bronchoscope 50 includes a source of illumination and a video imaging system (not explicitly shown) and is coupled to monitoring equipment 60, e.g., a video display, for displaying the video images received from the video imaging system of bronchoscope 50.

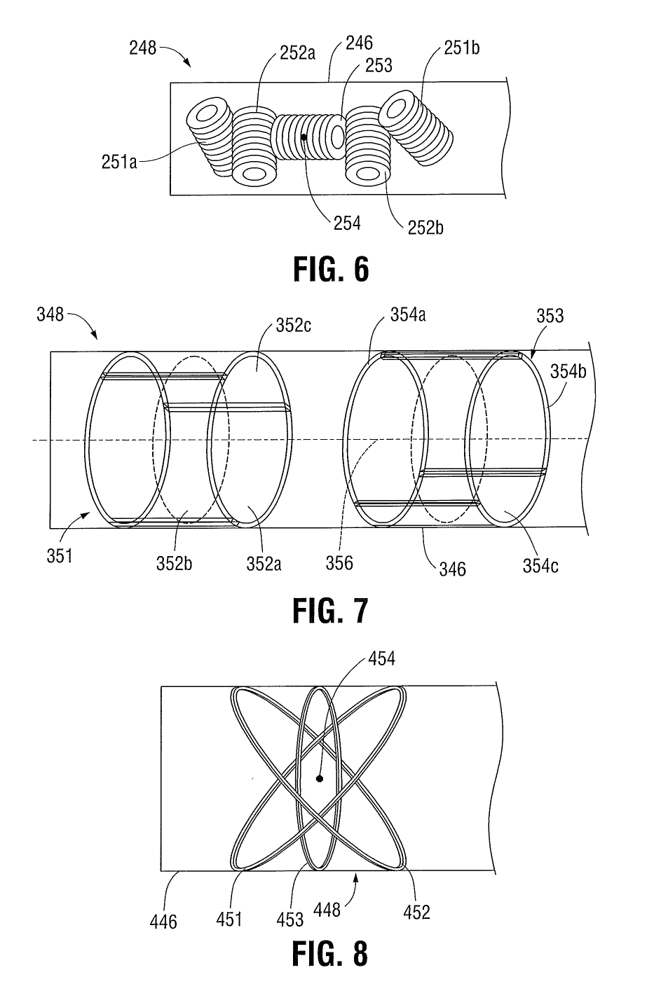

With respect to the navigation phase, a six degrees-of-freedom electromagnetic tracking system 70, e.g., similar to those disclosed in U.S. Pat. No. 6,188,355 and published PCT Application Nos. WO 00/10456 and WO 01/67035, the entire contents of each of which is incorporated herein by reference, or other suitable positioning measuring system, is utilized for performing registration and navigation, although other configurations are also contemplated. Tracking system 70 includes a tracking module 72, a plurality of reference sensors 74, and a transmitter mat 76. Tracking system 70 is configured for use with positioning assembly 90 and biopsy tool 100, as detailed below. Positioning assembly 90 includes a LG 92 having a steerable distal tip 93 incorporating a sensor 94, an EWC 96, and a handle 98. LG 92 and EWC 96 are configured for insertion through a working channel of bronchoscope 50 into the patient's airways (although LG 92 and EWC 96 may alternatively be used without bronchoscope 50) and are selectively lockable relative to one another via a locking mechanism 99. Steerable distal tip 93 of LG 92 may be configured for steering in any suitable fashion, e.g., using a plurality of steering wires (not shown) coupled between handle 98 and distal tip 93, to facilitate maneuvering distal tip 93 of LG 92 and EWC 96 through the patient's airways. Distal tip 93 of LG 92 may further define, at-rest, a linear, curved, or angled configuration, depending on a particular purpose. Sensor 94 is integrated with distal tip 93 of LG 92 and allows monitoring of the position and orientation of distal tip 93, in six degrees of freedom, relative to the reference coordinate system. Sensor 94 of LG 92 may be configured similar to any of the sensors detailed below (see FIGS. 6-8).

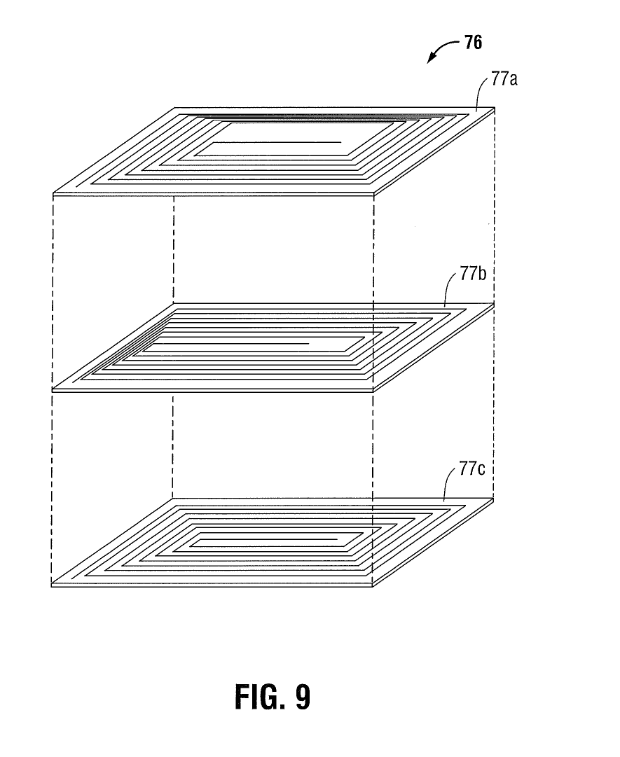

As shown in FIG. 1, transmitter mat 76 is positioned beneath patient “P.” The internal configuration of transmitter mat 76 will be detailed below with reference to FIG. 9. Transmitter mat 76 and the plurality of reference sensors 74 are interconnected with tracking module 72, which derives the location of each sensor 74 in six degrees of freedom. One or more of reference sensors 74 are attached to the chest of the patient “P.” The six degrees of freedom coordinates of reference sensors 74 are sent to computer 80 (which includes the appropriate software) where they are used to calculate a patient coordinate frame of reference. Registration, as detailed below, is generally performed by identifying locations in both the three-dimensional model and the patient's airways and measuring the coordinates in both systems. Further details of such a registration technique can be found in U.S. Patent Application Pub. No. 2011/0085720, the entire contents of which is incorporated herein by reference, although other suitable registration techniques are also contemplated. An exemplary embodiment of a transmitter mat 76, and the use thereof for determining location data, is detailed below.

In use, with respect to the navigation phase, LG 92 is inserted into EWC 96 such that sensor 94 projects from the distal end of EWC 96. LG 92 and EWC 96 are then locked together via locking mechanism 99. LG 92, together with EWC 96, are then inserted through bronchoscope 50 and into the airways of the patient “P,” with LG 92 and EWC 96 moving in concert with one another through bronchoscope 50 and into the airways of the patient “P.” Automatic registration is performed by moving LG 92 through the airways of the patient “P.” More specifically, data pertaining to locations of sensor 94 while LG 92 is moving through the airways is recorded using transmitter mat 76, reference sensors 74, and tracking module 72. A shape resulting from this location data is compared to an interior geometry of passages of the three-dimensional model generated in the planning phase, and a location correlation between the shape and the three-dimensional model based on the comparison is determined, e.g., utilizing the software on computer 80. In addition, the software identifies non-tissue space (e.g., air filled cavities) in the three-dimensional model. The software aligns, or registers, an image representing a location of sensor 94 of LG 92 with an image of the three-dimensional model based on the recorded location data and an assumption that LG 92 remains located in non-tissue space in the patient's airways. This completes the registration portion of the navigation phase.

Referring still to FIG. 1, once the planning phase has been completed, e.g., the target tissue has been identified and the pathway thereto selected, and registration has been completed, system 10 may be utilized to navigate LG 92 through the patient's airway to the target tissue. To facilitate such navigation, computer 80, monitoring equipment 60, and/or any other suitable display may be configured to display the three-dimensional model including the selected pathway from the current location of sensor 94 of LG 92 to the target tissue. Navigation of LG 92 to the target tissue using tracking system 70 is similar to that detailed below with respect to the navigation of biopsy tool 100 to the target tissue and, thus, is not detailed here for purposes of brevity.

Once LG 92 has been successfully navigated to the target tissue, completing the navigation phase, LG 92 may be unlocked from EWC 96 and removed, leaving EWC 96 in place as a guide channel for guiding biopsy tool 100 to the target tissue. Details of various embodiments of biopsy tools, along with the use of the same in the biopsy phase, are described below.

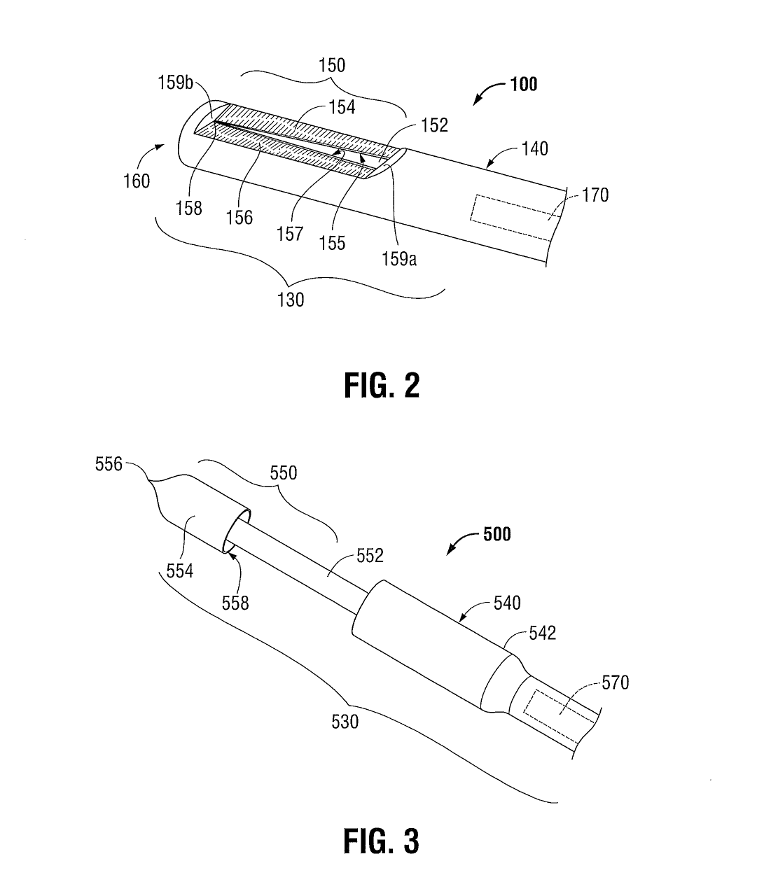

Referring now to FIG. 2, in conjunction with FIG. 1, one embodiment of a biopsy tool provided in accordance with the present disclosure for obtaining a tissue sample from the target tissue is shown generally identified by reference numeral 100. As detailed below, biopsy tool 100 is further configured for use in conjunction with tracking system 70 to facilitate navigation of biopsy tool 100 to the target tissue and/or tracking of biopsy tool 100 as it is manipulated relative to the target tissue to obtain the tissue sample. Although registration and navigation are detailed above with respect to LG 92 of positioning assembly 90, it is also envisioned that LG 92 be eliminated and biopsy tool 100 itself be utilized for registration and navigation, similarly as detailed above with respect to LG 92.

Biopsy tool 100, as best shown in FIG. 1, generally includes an elongated flexible body 110 interconnecting a proximal handle portion 120 and a rigid distal biopsy member 130. Proximal handle portion 120 is configured to facilitate manipulation of biopsy member 130, e.g., through bronchoscope 50 and EWC 96, and relative to tissue. Flexible body 110 is configured to enable insertion of biopsy tool 100 into a patient airways, e.g., through bronchoscope 50 and EWC 96 to the target tissue. Biopsy tool 100 is further configured to connect to a vacuum source “V” for applying suction at biopsy member 130, as will be detailed below.

With reference to FIG. 2, rigid distal biopsy member 130 includes a throat portion 140, a tissue-receiving portion 150, and a distal end cap 160. Throat portion 140 defines a generally cylindrical configuration and houses a sensor 170. Sensor 170, in conjunction with tracking system 70 (FIG. 1), enables tracking of biopsy member 130 of biopsy tool 100 as biopsy member 130 is advanced through the patient's airways, as detailed below. Thus, with additional reference to FIG. 1, computer 80, monitoring equipment 60, and/or any other suitable display may be configured to display the three-dimensional model and selected pathway, both of which were generated during the planning phase, along with the current location of sensor 170 of biopsy member 130 to facilitate navigation of biopsy member 130 to the target tissue and/or manipulation of biopsy member 130 relative to the target tissue. Various sensors suitable for use with biopsy member 130 for this purpose are detailed below (see FIGS. 6-8). Alternatively, biopsy tool 100 may not include a sensor and, rather, only LG 92 may be utilized for navigation and positioning. Distal end cap 160 of biopsy member 130 defines a generally blunt configuration, although distal end cap may alternatively be configured to facilitate tissue cutting.

Tissue-receiving portion 150 is configured to receive a tissue sample therethrough and into the generally hollow interior of biopsy member 130. More specifically, tissue-receiving portion 150 includes a window 152 configured to receive tissue therethrough. Window 152 is defined by first and second longitudinally-extending faces 154, 156. Faces 154, 156 are angled into the interior of tissue-receiving portion 150 and are oriented to define an acute interior angle therebetween, e.g., a generally “V”-shaped configuration. Faces 154, 156 each includes a sharpened cutting edge 155, 157, respectively, disposed on one side of window 152. As a result of their positioning and orientation, faces 154, 156 are at least partially recessed relative to throat portion 140 and distal end cap 160 of biopsy member 130. Thus, proximal and distal shoulders 159a, 159b, respectively, are defined on either end of tissue-receiving portion 150. Faces 154, 156 are further oriented relative to one another such that edges 155, 157 increasingly approximate one another in the proximal-to-distal direction, ultimately culminating at an apex point 158 adjacent distal shoulder 159b. This feature facilitates dynamic tissue cutting, as detailed below.

Referring to FIGS. 1-2, in use, once the planning and navigation phases have been completed, and LG 92 removed from EWC 96, biopsy tool 100 may be inserted through bronchoscope 50 and EWC 96 to the target tissue. Sensor 170 of biopsy member 130, in conjunction with tracking system 70, as mentioned above, enables tracking of sensor 170 as it is advanced through the patient's airways. Thus, even after biopsy member 130 is extended distally from EWC 96, the position of biopsy member 130 can be tracked, thus permitting navigation of biopsy member 130 to and/or manipulation of biopsy member 130 relative to the target tissue to ensure proper positioning of biopsy member 130 relative to the target tissue and allowing certain tissue structures adjacent the target tissue to be avoided. Details of tracking and navigating using suitable sensors and tracking system 70 will be described in greater detail below, following the description of the various embodiments thereof.

Once biopsy member 130 of biopsy tool 100 is positioned as desired, vacuum source “V” may be activated to apply suction at window 152 of tissue-receiving portion 150 of biopsy member 130 to suction tissue into the interior of tissue-receiving portion 150. As a sample of tissue is suctioned through window 152, the sample is cut away from laterally surrounding tissue via the urging of tissue into contact with edges 155, 157, e.g., as a result of the suction force applied to tissue. Once the tissue sample has been at least partially received within the interior of tissue-receiving portion 150, biopsy member 130 may be translated proximally relative to tissue, e.g., via grasping and translating proximal handle portion 120 proximally, such that the tissue sample is completely severed from surrounding tissue. This severing of the tissue sample is aided by the relative movement of approximating edges 155, 157 and apex point 158 relative to and through tissue. Upon receiving and fully separating the tissue sample from surrounding tissue, biopsy tool 100 may be withdrawn from the patient's airways and the tissue sample retrieved from biopsy tool 100 for testing. It is also contemplated that multiple sample be taken with biopsy tool 100, e.g., at the same location or various different locations, prior to withdrawal

Referring now to FIG. 3, another embodiment of a biopsy tool provided in accordance with the present disclosure for obtaining a tissue sample from the target tissue is shown generally identified by reference numeral 500. Similarly as detailed above with respect to the previous embodiment, biopsy tool 500 is configured for use in conjunction with tracking system 70 (FIG. 1) to facilitate navigation of biopsy tool 500 to the target tissue and/or tracking of biopsy tool 500 as it is manipulated relative to the target tissue to obtain the tissue sample.

Biopsy tool 500 generally includes an elongated flexible body (not explicitly shown, similar to body 110 of biopsy tool 100 (FIG. 1) interconnecting a proximal handle portion (not explicitly shown, similar to handle portion 120 of biopsy tool 100 (FIG. 1) and a distal biopsy member 530. The handle portion (not shown) is manually operable to manipulate biopsy member 530. The flexible body (not shown) is configured to enable insertion of biopsy tool 500 into a patient airways, e.g., through bronchoscope 50 and EWC 96 to the target tissue (See FIG. 1).

Distal biopsy member 530 includes an outer member 540 and an inner member 550 that is both translatable and rotatable relative to outer member 540. Outer member 540 defines a generally hollow configuration and includes an enlarged body portion 542. Body portion 542 is configured to at least partially receive distal end cap 554 of inner member 550 when inner member 550 is disposed in the retracted position, as will be detailed below. Outer member 540 is further configured to house a sensor 570 therein. Similarly as detailed above with respect to the previous embodiment, sensor 570, in conjunction with tracking system 70 (FIG. 1), enables tracking of biopsy member 530 of biopsy tool 500 as biopsy member 530 is advanced through the patient's airways, as detailed below. Various sensors suitable for use with biopsy member 530 for this purpose are detailed below (see FIGS. 6-8). Alternatively, biopsy tool 500 may not include a sensor and, rather, only LG 92 (FIG. 1) may be utilized for navigation and positioning.

Inner member 550 includes a shaft 552 and a distal end cap 554 mounted at the distal end of shaft 552. Inner member 550 is translatable relative to outer member 540 between a retracted position, wherein shaft 552 is disposed within outer member 540 and wherein distal end cap 554 is at least partially disposed within enlarged body portion 542 of outer member 540, and an extended position, wherein distal end cap 554 extends and is distally-spaced from outer member 540 (as shown in FIG. 3). Distal end cap 554 includes a sharpened tip 556 configured for facilitate puncturing and penetrating tissue upon advancement of distal end cap 554 into tissue, and a sharpened proximal rim 558 configured to core tissue upon simultaneous rotation and proximal translation of distal end cap 554 relative to tissue. Distal end cap 554 may further define a generally hollow interior and an open proximal end configured to receive a tissue sample therein, e.g., once the tissue sample has been cored from surrounding tissue.

With additional reference to FIG. 1, in use, once the planning and navigation phases have been completed, and LG 92 removed from EWC 96, biopsy tool 500, with inner member 550 disposed in the retracted position, may be inserted through bronchoscope 50 and EWC 96 to the target tissue. Sensor 570 of biopsy member 530, in conjunction with tracking system 70, as mentioned above, enable tracking of sensor 570, thus permitting navigation of biopsy member 530 to and/or manipulation of biopsy member 530 relative to the target tissue to ensure proper positioning of biopsy member 530 relative to the target tissue and allowing certain tissue structures adjacent the target tissue to be avoided. Details of tracking and navigating using suitable sensors and tracking system 70 will be described in greater detail below, following the description of the various embodiments thereof.

Once biopsy member 530 of biopsy tool 500 is positioned as desired, e.g., adjacent target tissue to be sampled, inner member 550, lead by sharpened tip 556 of distal end cap 554, is translated distally from the retracted position to the extended position to penetrate the target tissue. Once advanced to a sufficient depth within the target tissue, inner member 550 may be returned to the retracted position relative to outer member 540 while being simultaneously rotated relative to outer member 540 such that the tissue that was positioned between inner and outer members 550, 540, respectively, is cored or separated from surrounding tissue using sharpened proximal rim 558 and is retained within the hollow interior of distal end cap 554 and/or outer member 540. In some embodiments, biopsy tool 500 may further be configured to connect to the vacuum source “V” (FIG. 1) to facilitate obtaining a tissue sample. Upon receiving and fully separating the tissue sample(s) from surrounding tissue, biopsy tool 500 may be withdrawn from the patient's airways and the tissue sample retrieved from biopsy tool 500 for testing.

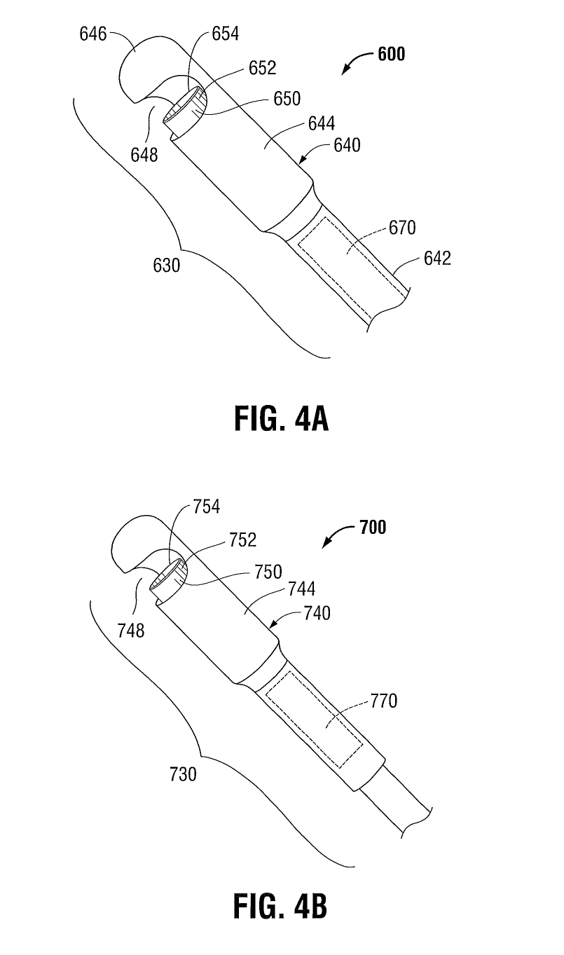

Referring now to FIG. 4A, another embodiment of a biopsy tool provided in accordance with the present disclosure for obtaining a tissue sample from the target tissue is shown generally identified by reference numeral 600. Similarly as detailed above with respect to the previous embodiment, biopsy tool 600 is configured for use in conjunction with tracking system 70 (FIG. 1) to facilitate navigation of biopsy tool 600 to the target tissue and/or tracking of biopsy tool 600 as it is manipulated relative to the target tissue to obtain the tissue sample.

Biopsy tool 600 generally includes an elongated flexible body (not explicitly shown, similar to body 110 of biopsy tool 100 (FIG. 1)) interconnecting a proximal handle portion (not explicitly shown, similar to handle portion 120 of biopsy tool 100 (FIG. 1) and a distal biopsy member 630. The handle portion (not shown) is manually operable to manipulate biopsy member 630. The flexible body (not shown) is configured to enable insertion of biopsy tool 600 into a patient airways, e.g., through bronchoscope 50 and EWC 96 to the target tissue (See FIG. 1). Biopsy tool 600 is further configured to connect to a vacuum source “V” (FIG. 1) for applying suction at biopsy member 630, as will be detailed below.

Distal biopsy member 630 includes an outer member 640 and an inner member 650 that is fixedly disposed within outer member 640. Outer member 640 defines a generally hollow configuration and includes a body portion 642 and a head portion 644. Body portion 642 is configured to house a sensor 670 therein. Similarly as detailed above with respect to the previous embodiments, sensor 670, in conjunction with tracking system 70 (FIG. 1), enables tracking of biopsy member 630 of biopsy tool 600 as biopsy member 630 is advanced through the patient's airways, as detailed below. Various sensors suitable for use with biopsy member 630 for this purpose are detailed below (see FIGS. 6-8). Alternatively, biopsy tool 600 may not include a sensor and, rather, only LG 92 (FIG. 1) may be utilized for navigation and positioning.

Continuing with reference to FIG. 4A, head portion 644 of outer member 640 includes a blunt distal cap 646 and a mouth 648 defined through a lateral wall of outer member 640 towards the distal end thereof. Mouth 648 provides access to the hollow interior of outer member 640 and inner member 650 which, as mentioned above, is fixedly disposed within outer member 640.

Inner member 650 defines a generally cylindrical configuration and includes a open distal end 652 defining a sharpened rim 654. Open distal end 652 of inner member 650 terminates in the vicinity of mouth 648 of outer member 640 such that sharpened rim 654 is exposed adjacent mouth 648. Further, inner member 650 is coupled to the vacuum source “V” (FIG. 1) for applying suction at open distal end 652 of inner member 650 to suction a tissue sample through mouth 648 and into open distal end 652 of inner member 650, while the tissue sample is severed from surrounding tissue via sharpened rim 654.

With additional reference to FIG. 1, in use, once the planning and navigation phases have been completed, and LG 92 removed from EWC 96, biopsy tool 600 may be inserted through bronchoscope 50 and EWC 96 to the target tissue. Sensor 670 of biopsy member 130, in conjunction with tracking system 70, as mentioned above, enables tracking of sensor 670, thus permitting navigation of biopsy member 630 to and/or manipulation of biopsy member 630 relative to the target tissue to ensure proper positioning of biopsy member 630 relative to the target tissue and allowing certain tissue structures adjacent the target tissue to be avoided. Details of tracking and navigating using suitable sensors and tracking system 70 will be described in greater detail below, following the description of the various embodiments thereof.

Once biopsy member 630 of biopsy tool 600 is positioned as desired, mouth 648 is oriented towards target tissue and vacuum source “V” (FIG. 1) is activated to apply suction adjacent mouth 648 to suction a tissue sample through mouth 648 and into open distal end 652 of inner member 650. As a sample of tissue is suctioned through mouth 648, the tissue sample is severed from surrounding tissue via sharpened rim 654. Upon receiving and fully separating the tissue sample(s) from surrounding tissue, biopsy tool 600 may be withdrawn from the patient's airways and the tissue sample retrieved from biopsy tool 600 for testing.

Turning to FIG. 4B, another embodiment of a biopsy tool provided in accordance with the present disclosure for obtaining a tissue sample from the target tissue is shown generally identified by reference numeral 700. Biopsy tool 700 is similar to biopsy tool 600 (FIG. 4A) and, thus, only the differences therebetween will be described in detail below for purposes of brevity.

Biopsy tool 700 generally includes an elongated flexible body (not explicitly shown) interconnecting a proximal handle portion (not explicitly shown) and a distal biopsy member 730. Biopsy tool 700 is further configured to connect to a vacuum source “V” (FIG. 1) for applying suction at biopsy member 730, as will be detailed below.

Distal biopsy member 730 includes an outer member 740 and an inner member 750 that is disposed within and rotatably coupled to outer member 740, thus enabling rotation of inner member 750 relative to outer member 740. Outer member 740 is configured to house a sensor 770 therein and includes a head portion 744 defining a mouth 748. Inner member 750 defines a generally cylindrical configuration and includes a open distal end 752 defining a sharpened rim 754.

In use, once biopsy member 730 of biopsy tool 700 is positioned as desired, mouth 748 is oriented towards target tissue and vacuum source “V” (FIG. 1) is activated to apply suction adjacent mouth 748 to suction a tissue sample through mouth 748 and into inner member 750. As a sample of tissue is suctioned through mouth 748, the tissue sample is severed from surrounding tissue via sharpened rim 754. Severing the tissue sample from surrounding tissue may be aided by selectively rotating inner member 750 relative to outer member 740 while applying suction. Ultimately, biopsy tool 700 may be withdrawn from the patient's airways and the tissue sample(s) retrieved from biopsy tool 700 for testing.

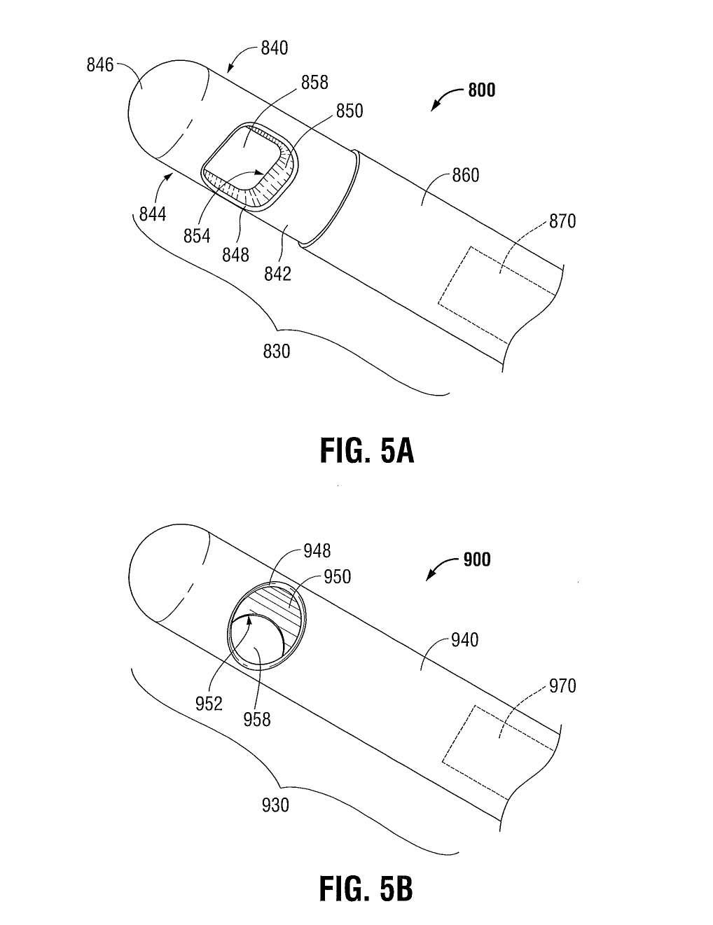

Referring now to FIG. 5A, another embodiment of a biopsy tool provided in accordance with the present disclosure for obtaining a tissue sample from the target tissue is shown generally identified by reference numeral 800. Similarly as detailed above with respect to the previous embodiments, biopsy tool 800 is configured for use in conjunction with tracking system 70 (FIG. 1) to facilitate navigation of biopsy tool 800 to the target tissue and/or tracking of biopsy tool 800 as it is manipulated relative to the target tissue to obtain the tissue sample.

Biopsy tool 800 generally includes an elongated flexible body (not explicitly shown, similar to body 110 of biopsy tool 100 (FIG. 1)) interconnecting a proximal handle portion (not explicitly shown, similar to handle portion 120 of biopsy tool 100 (FIG. 1)) and a distal biopsy member 830. The handle portion (not shown) is manually operable to manipulate biopsy member 830. The flexible body (not shown) is configured to enable insertion of biopsy tool 800 into a patient airways, e.g., through bronchoscope 50 and EWC 96 to the target tissue (See FIG. 1). Biopsy tool 800 is further configured to connect to a vacuum source “V” (FIG. 1) for applying suction at biopsy member 830, as will be detailed below.

Distal biopsy member 830 includes an outer member 840, an inner member 850 that is fixedly disposed within outer member 840, and a sleeve 860 that is disposed about outer member 840. Outer member 840 defines a generally hollow configuration and includes a body portion 842 and a head portion 844. Body portion 842 is configured to house a sensor 870, similarly as detailed above with respect to the previous embodiments.

Head portion 844 of outer member 840 includes a blunt distal cap 846 and a mouth 848 defined through a lateral wall of outer member 840 towards the distal end thereof. Mouth 848 provides access to the hollow interior of outer member 840 and inner member 850 which, as mentioned above, is fixedly disposed within outer member 840.

Inner member 850 is fixedly disposed within outer member 840 and, similar to outer member 840, includes a mouth 858 defined through a lateral wall thereof towards the distal end thereof. Mouth 858 defines a sharpened rim 854 configured to facilitate tissue cutting and is positioned adjacent mouth 848 of outer member 840 such that sharpened rim 854 is exposed adjacent mouth 848. Further, inner member 850 is coupled to the vacuum source “V” (FIG. 1) for applying suction at mouth 858.

With additional reference to FIG. 1, in use, once the planning and navigation phases have been completed, and LG 92 removed from EWC 96, biopsy tool 800 may be inserted through bronchoscope 50 and EWC 96 to the target tissue. Sensor 870 of biopsy member 830, in conjunction with tracking system 70, as mentioned above, enables tracking of sensor 870, thus permitting navigation of biopsy member 830 to and/or manipulation of biopsy member 830 relative to the target tissue to ensure proper positioning of biopsy member 830 relative to the target tissue and allowing certain tissue structures adjacent the target tissue to be avoided. Details of tracking and navigating using suitable sensors and tracking system 70 will be described in greater detail below, following the description of the various embodiments thereof.

Once biopsy member 830 of biopsy tool 800 is positioned as desired, mouth 848 is oriented towards target tissue and vacuum source “V” (FIG. 1) is activated to apply suction adjacent mouth 848 to suction a tissue sample through mouth 848 and into mouth 858 of inner member 850. As a sample of tissue is suctioned through mouth 848 and into mouth 858, the tissue sample is severed from surrounding tissue via sharpened rim 854. Severing the tissue sample from surrounding tissue may be aided by selectively translating biopsy member 830 proximally relative to tissue while applying suction. Upon receiving and fully separating the tissue sample(s) from surrounding tissue, biopsy tool 800 may be withdrawn from the patient's airways and the tissue sample retrieved from biopsy tool 800 for testing.

Turning to FIG. 5B, another embodiment of a biopsy tool provided in accordance with the present disclosure for obtaining a tissue sample from the target tissue is shown generally identified by reference numeral 900. Biopsy tool 900 is similar to biopsy tool 800 (FIG. 5A) and, thus, only the differences therebetween will be described in detail below for purposes of brevity.

Biopsy tool 900 generally includes an elongated flexible body (not explicitly shown) interconnecting a proximal handle portion (not explicitly shown) and a distal biopsy member 930. Biopsy tool 900 is further configured to connect to a vacuum source “V” (FIG. 1) for applying suction at biopsy member 930, as will be detailed below.

Distal biopsy member 930 includes an outer member 940 and an inner member 950 that is disposed within and rotatably coupled to outer member 940. Outer member 940 is configured to house a sensor 970 and defines a mouth 948 through a lateral wall thereof towards the distal end thereof. Inner member 950, similar to outer member 940, includes a mouth 958 defined through a lateral wall thereof towards the distal end thereof. Mouth 958 defines a sharpened rim 954 configured to facilitate tissue cutting and is positioned adjacent mouth 948 of outer member 940. Inner member 950 is rotatable relative to outer member 940 to thereby vary the relative positioning of mouths 948, 958, e.g., between an aligned position, a partially overlapping position, and a fully occluded position Inner member 950 is coupled to the vacuum source “V” (FIG. 1) for applying suction at mouth 958.

With additional reference to FIG. 1, in use, Once biopsy member 930 of biopsy tool 900 is positioned as desired, inner member 950 is rotated such that mouths 948, 958 are aligned with one another, and vacuum source “V” (FIG. 1) is activated to apply suction adjacent mouth 958 to suction a tissue sample through mouths 948, 958 and into inner member 950. Once a sample of tissue is suctioned through mouths 948, 958 and into inner member 950, inner member 950 is rotated relative to outer member 940 such that mouths 948, 958 are moved towards an occluded position. As mouths 948, 958 are moved towards the occluded position, tissue disposed therebetween is cut via sharpened rim 854, thereby severing the tissue sample from surrounding tissue. Upon receiving and fully separating the tissue sample(s) from surrounding tissue, biopsy tool 900 may be withdrawn from the patient's airways and the tissue sample retrieved from biopsy tool 900 for testing.

Turning now to FIGS. 6-8, in conjunction with FIG. 1, various different sensors 248, 348, 448 (FIGS. 6-8, respectively) configured for use as the sensor of any of the biopsy tools detailed herein and/or sensor 94 of LG 92 are described. Referring to FIG. 6, sensor 248 is shown. Sensor 248 includes a plurality of field component sensor elements 251a, 251b, 1252a, 252b, 253. Each sensor element 251a, 251b, 252a, 252b, 253 is formed as a coil and arranged for sensing a different component of an electromagnetic field generated by transmitter mat 76 (FIG. 9). More specifically, first and second pairs of sensor elements 251a, 251b and 252a, 252b are arranged within sensor housing 246 such that the respective elements 251a, 251b and 252a, 252b of each pair are equidistant from a common reference point 254, while sensor element 253 is centered about reference point 254. Although shown in FIG. 6 as collinearly disposed, other configurations of sensor elements 251a, 251b, 1252a, 252b, 253 are also contemplated. Further, as opposed to providing five sensor elements 251a, 251b, 1252a, 252b, 253 wherein sensor element 253 is centered about the reference point 254, six sensors may be provide, e.g., wherein sensor element 253 is provided as a pair of elements disposed equidistant from reference point 254. The above-described configuration of sensor 248 enables transmitter mat 76 and the plurality of reference sensors 74 (FIG. 1), together with tracking module 72 and computer 80 (FIG. 1), to derive the location of sensor 248 in six degrees of freedom, as detailed below, and as further detailed in U.S. Pat. No. 6,188,355 and published PCT Application Nos. WO 00/10456 and WO 01/67035, previously incorporated herein by reference.

With reference to FIG. 7, sensor 348 is shown including two sensor components 351, 353 arranged within sensor housing 346, each component 351, 353 including three sensor elements 352a, 352b, 352cand 354a, 354b, 354c, respectively. Each sensor element 352a, 352b, 352c and 354a, 354b, 354c is configured as a flat rectangular coil, e.g., including a plurality of turns of conducting wire, bent to define an arcuate shape. As such, the elements 352a, 352b, 352c and 354a, 354b, 354c combine to define first and second generally cylindrical components 351, 353. Components 351, 353 are centered about reference axis 356 and positioned such that each of elements 352a, 352b, 352c and 354a, 354b, 354c are equidistant from reference axis 356 and such that each of elements 352a, 352b, 352c of component 351 are oriented 180 degrees offset as compared to corresponding elements 354a, 354b, 354c, respectively, of component 353. Thus, similarly as with sensor 248 (FIG. 6), sensor 348 enables transmitter mat 76 and the plurality of reference sensors 74 (FIG. 1), together with tracking module 72 and computer 80 (FIG. 1), to derive the location of sensor 348 in six degrees of freedom.

Turning to FIG. 8, sensor 448 includes three coils 451, 452, 453. Coils 451 and 452, 453 are angled relative to housing 446, while coil 453 is circumferentially disposed within housing 446. Coils 451, 452, 453 are oriented to lie in perpendicular planes relative to one another and share a common center reference point 454. By sharing a common center reference point 454, each portion of each coil 451, 452, 453 is equidistant from center reference point 454. Further, this configuration, e.g., wherein coils share a common center reference point 454 rather than being longitudinally displaced relative to one another, allows for the longitudinal dimension of sensor 448 to be minimized. Such a configuration still, however, enables transmitter mat 76 and the plurality of reference sensors 74 (FIG. 1), together with tracking module 72 and computer 80 (FIG. 1), to derive the location of sensor 448 in six degrees of freedom.

Referring to FIG. 9, in conjunction with FIG. 1, an embodiment of the internal configuration of transmitter mat 76 of tracking system 70 (FIG. 1) is shown, although other suitable configurations are also contemplated. Transmitter mat 76 is a transmitter of electromagnetic radiation and includes a stack of three substantially planar rectangular loop antennas 77a, 77b, 77c configured to connected to drive circuitry (not shown).

Antenna 77a is skewed in a first horizontal direction (when the transmitter mat 76 is horizontal) in that the loops on one side of the antenna 77a are closer together than the loops on the opposite side. As a result, antenna 77a creates a magnetic field that is stronger on the side where the loops are close together than on the opposite side. By measuring the strength of the current induced by antenna 77a in the sensor assembly, e.g., sensor assembly 145 of biopsy tool 100 (FIG. 3) or sensor 94 of LG 92 (FIG. 1), it can be determined where the sensor assembly is located in the first direction over antenna 77a.

Antenna 77b is similar to antenna 77a except that antenna 77b is skewed in an second horizontal direction that is perpendicular to the first direction. By measuring the strength of the current induced by antenna 77b in the sensor assembly, it can be determined where the sensor assembly is located in the second direction over antenna 77b.

Antenna 77c defines a uniform, i.e., un-skewed, configuration. Thus, antenna 77c creates a uniform field that naturally diminishes in strength in a vertical direction when the transmitter mat 76 is horizontal. By measuring the strength of the field induced in the sensor assembly, it can be determined how far the sensor assembly is located above antenna 77c.

In order to distinguish one magnetic field from another, the fields of antennae 77a, 77b, 77c are generated using independent frequencies. For example, antenna 77a may be supplied with alternating current oscillating at 2.5 kHz, antenna 77b may be supplied with alternating current oscillating at 3.0 kHz, and antenna 77c may be supplied with alternating current oscillating at 3.5 kHz, although other configurations are also contemplated. As a result of using independent frequencies, each of the sensor components of the sensor assembly (see FIGS. 6-8, for example) will have a different alternating current signal induced in its coils.

Referring additionally to FIG. 1, in use, signal generators and amplifiers of the driving circuitry (not shown) associated with tracking system 70 are utilized to drive each of antennas 77a, 77b, 77c of transmitter mat 76 at their corresponding frequencies. The electromagnetic waves generated by transmitter mat 76 are received by the various sensor elements of the sensor assembly e.g., the sensor elements of sensors 248, 348, 448 (FIGS. 6-8, respectively) configured for use any of the biopsy tools provided herein or sensor 94 of LG 92, and are converted into electrical signals that are sensed via reference sensors 74. Tracking system 70 further includes reception circuitry (not shown) that has appropriate amplifiers and A/D converters that are utilized to receive the electrical signals from reference sensors 74 and process these signals to determine and record location data of the sensor assembly. Computer 80 may be configured to receive the location data from tracking system 70 and display the current location of the sensor assembly on the three-dimensional model and relative to the selected pathway generated during the planning phase, e.g., on computer 80, monitoring equipment 60, or other suitable display. Thus, navigation of the biopsy tool and/or LG 92 to the target tissue and/or manipulation of the biopsy tool relative to the target tissue, as detailed above, can be readily achieved.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A biopsy tool, comprising:
an elongated flexible body defining a distal end;
a distal biopsy member disposed at the distal end of the elongated flexible body, the distal biopsy member incorporating a sensor assembly including at least one location sensor configured to enable detection of a location of the sensor assembly within a patient's airways, the distal biopsy member having a tissue-receiving portion defining a window and including first and second longitudinally-extending faces disposed on either side of the window, the faces angled inwardly and towards one another to define an acute interior angle therebetween, each face defining a sharpened cutting edge, the sharpened cutting edges disposed on either side of the window, the faces positioned such that the sharpened cutting edges increasingly approximate one another in the proximal-to-distal direction and culminate at an apex point.

2. The biopsy tool according to claim 1, wherein the tissue-receiving portion of the distal biopsy member is recessed relative to a body of the distal biopsy member to define proximal and distal shoulders at proximal and distal ends of the tissue-receiving portion.

3. The biopsy tool according to claim 1, wherein the distal biopsy member is configured to connect to a vacuum source for applying suction adjacent the window.

4. A biopsy tool, comprising:
an elongated flexible body defining a distal end;
a distal biopsy member disposed at the distal end of the elongated flexible body, the distal biopsy member incorporating a sensor assembly including at least one location sensor configured to enable detection of a location of the sensor assembly within a patient's airways, the distal biopsy member including:
an outer member defining a hollow configuration; and
an inner member including a shaft and a distal end cap, the inner member slidable relative to the outer member between a retracted position, wherein the shaft is disposed within the outer member and the distal end cap is at least partially disposed within outer member, and an extended position, wherein the distal end cap and the shaft extend distally from the outer member such that the distal end cap is distally-spaced from the outer member, the distal end cap defining a sharpened distal tip configured to facilitate tissue penetration and a sharpened proximal rim configured to facilitate cutting tissue disposed between the distal end cap and the outer member upon return of the inner member towards the retracted position.

5. The biopsy tool according to claim 4, wherein the inner member is rotatable relative to the outer member to further facilitate cutting tissue disposed between the distal end cap and the outer member upon return of the inner member towards the retracted position.

6. The biopsy tool according to claim 4, wherein the distal end cap defines a hollow interior configured to receive a portion of a tissue sample therein.

7. A biopsy tool, comprising:
an elongated flexible body defining a distal end;
a distal biopsy member disposed at the distal end of the elongated flexible body, the distal biopsy member incorporating a sensor assembly including at least one location sensor configured to enable detection of a location of the sensor assembly within a patient's airways, the distal biopsy member including:
an outer member including a head portion defining a distal end cap and having a mouth extending through a lateral wall of the head portion towards the distal end cap; and
an inner member disposed within the outer member, the inner member defining an open distal end having a sharpened rim positioned adjacent the mouth of the outer member.

8. The biopsy tool according to claim 7, wherein the inner member is fixed relative to the outer member.

9. The biopsy tool according to claim 7, wherein the inner member is rotatable relative to the outer member.

10. The biopsy tool according to claim 7, wherein the distal biopsy member is configured to connect to a vacuum source for applying suction adjacent the open distal end of the inner member.

11. A biopsy tool, comprising:
an elongated flexible body defining a distal end;
a distal biopsy member disposed at the distal end of the elongated flexible body, the distal biopsy member incorporating a sensor assembly including at least one location sensor configured to enable detection of a location of the sensor assembly within a patient's airways, the distal biopsy member including:
an outer member including a head portion defining a distal end cap and having a first mouth extending through a lateral wall of the head portion towards the distal end cap; and
an inner member disposed within the outer member, the inner member defining a second mouth extending through a lateral wall of the inner member and positioned adjacent the first mouth, the inner member including a sharpened rim disposed about the second mouth.

12. The biopsy tool according to claim 11, wherein the inner member is fixed relative to the outer member.

13. The biopsy tool according to claim 11, wherein the inner member is rotatable relative to the outer member to move the first and second mouths at least between an aligned position, a partially overlapping position, and an occluded position.

14. The biopsy tool according to claim 11, wherein the distal biopsy member is configured to connect to a vacuum source for applying suction adjacent the second mouth of the inner member.
